(19) United States
(12) Patent Application Publication
(10) Pub. No.: US 2024/0130758 A1
(43) Pub. Date: Apr. 25, 2024

(54) SURGICAL SYSTEM FOR MINIMALLY INVASIVE TREATMENT OF A TRIGGER FINGER

(71) Applicant: Centre Hospitalier Universitaire Vaudois (CHUV), Lausanne (CH)

(72) Inventors: Debora Schivo, Sion (CH); Sebastien Durand, Lausanne (CH); Didier Maillefer, Lausanne (CH); Severino Tehrani, St-Legier-Ls Chiesaz (CH)

(73) Assignee: Centre Hospitalier Universitaire Vaudois (CHUV), Lausanne (CH)

(21) Appl. No.: 18/277,939

(22) PCT Filed: Feb. 16, 2022
(86) PCT No.: PCT/EP2022/053733

§ 371 Date: Aug. 17, 2023

(30) Foreign Application Priority Data
Feb. 19, 2021 (EP) 21158100.4

(51) Int. Cl. A61B 17/3211; A61B 17/00

(52) U.S. Cl. CPC A61B 17/3211; A61B 17/00234; A61B 2017/32113

(57) ABSTRACT

The invention relates to a surgical device to be used in orthopaedic treatment. In particular, the present invention relates to a surgical system for minimally invasive treatment of a trigger finger.

### BACKGROUND OF THE INVENTION

The invention relates to a surgical device to be used in orthopaedic treatment. In particular, the present invention relates to a surgical system for minimally invasive treatment of a trigger finger.

Trigger finger or stenosing tenosynovitis is a common medical disorder, which is often caused by frequent repetitive gripping actions or trauma. Typically, it occurs at the level of the first annual ligament (A1 pulley) of the fingers. It is characterised by the catching or locking of the involved flexor tendon. When a tendon is inflamed or swollen, it may not fit properly into the tendon sheath that surrounds and supports it. In other words, one of the fingers gets stuck in a bent position and straightens with a snapping motion.

The signs and symptoms of trigger finger can vary depending on the severity, including stiffness, numbness, weakness, pain, clicking sensation felt during the movement of the affected finger and tenderness or a bump in the palm at the base of the finger. It can further progress into a stage where the finger becomes immobilised and dysfunctional. In more serious cases, a partial deformation of the flexor tendon occurs.

Although certain groups of people are at higher risk of developing a trigger finger more easily, e.g., diabetes patients, people with occupations and hobbies involving repetitive hand use, elderly people or women, it is a relatively common medical condition.

Due to the complex anatomy and vulnerability of the hand, a reliable and fail-proof device is desirable. Even a small injury inflicted during a treatment could prolong the recovery or result in a permanent damage.

At an early stage, the condition is most commonly treated by direct steroid injection in the area of the affected pulley to reduce inflammation. However, if the injections are no longer effective, a surgical intervention becomes inevitable.

A conventional treatment to release the constricted tissue is open surgery. Open surgery has multiple drawbacks. First, the open surgery requires a relatively large incision over the affected pulley, which often leaves a visible scar. The subsequent healing process of the hand is long, painful and inconvenient for a patient. Furthermore, the operation requires a highly trained medical professional and an operation hall, which increases the expenses altogether. The success rate of the open surgery may depend on the experience of the surgeon.

An endoscopic surgery allows to minimise the invasiveness of the surgery but the equipment to perform the endoscopy is in most cases expensive.

Alternatively, the minimally invasive treatment of a trigger finger continues to gain in importance. However, a subcutaneous incision without endoscopic guidance tends to significantly lower the accuracy of the operation. The disadvantage of not being able to observe the tissue and the blade requires the operation to be performed blindly. Thus, the affected pulley is searched through an exploration relying on the instinct or experience of the surgeon. During the exploration, tissues and nerves nearby can be injured. Since the tissue under the skin is not visible, it is also not easy to ascertain whether the affected tissue has been found and severed as desired. Therefore, a complication or even a relapse of the medical condition is possible.

On the other hand, minimising the incision can equally minimise the damage to the nerves around the incision and thereby reduce the scar size and the recovery time. Furthermore, the pain level and the inconvenience after the treatment are considerably less compared to an open surgery. Minimally invasive surgery is thus desirable, but more safe and reliable devices and operation methods are required.

There are known devices for minimally invasive treatment of a trigger finger. However, the existing techniques possess disadvantages, e.g., associated with the safety or usability of the operation.

CN 209713070 U discloses a tenosynovitis scalpel assembly which comprises a scalpel body in a hollow tube shape. The blade body with a blade edge protected by the inner core sleeved into the body enters the skin. Upon reaching the lesion, the tissue is cut with the external blade, which is exposed by pulling out the protective inner core in a pulling motion. The pulling motion is not performed in a particularly controlled manner. There can also be uncertainty whether the blade is engaging the tissue.

CN 110338885 A discloses a surgical device for tenosynovitis comprising an insertion part and a cutting part. A needle located at the distal end of the device may pose a severe risk to the tissues upon entering the skin during a tenosynovitis surgery.

CN 106037888 A relates to a guiding-type cutting knife assembly and a guiding device thereof. The end of the guiding portion has a slope, which separates the flexor tendon tissue.

CN 104883988 A discloses an endoscopic surgical device having a slotted clear cannula, a blade and a housing, wherein the cannula is attached to the housing, and wherein the blade is enclosed in the housing and is slidable into the cannula. It requires endoscopic components to visualise the target tissue.

WO 2010/030872 A2 discloses a surgical instrument for use in releasing tissue within a body. The instrument comprises a first end made to facilitate inserting the surgical instrument into the body and also a blade member that is made to slide and move relative to the tube along a slot positioned along the tube's longitudinal axis. A relatively large incision is required for the blade member to reach the constricted tissue.

US 2006/190021 A1 discloses a cutting device for making subcutaneous incisions. It comprises a trocar and a tubular blade sub-assembly, which cuts through an affected pulley.

Accordingly, developing a new surgical instrument device and a corresponding surgical method to decrease the likelihood of complications when inserting the instrument, locating and releasing the affected tissue is desirable. The objective is, among others, to overcome drawbacks associated with devices of the prior art.

### SUMMARY OF THE INVENTION

The present invention satisfies the need for an improved surgical instrument for minimally invasive treatment of a trigger finger and the need for an improved method of using a surgical instrument to treat a trigger finger in a patient.

Terms such as “distal” and “proximal” are employed herein as generally used in the field, i.e. with the “distal” direction extending from a handle of the device towards an end of the device inserted into the patient and/or towards the pulley in use. Accordingly, the “proximal” direction extends away from the distal end and towards the handle and/or away from the pulley in use.

According to a first configuration of the present invention, a surgical system for minimally invasive treatment of a trigger finger comprises a handle with a lumen provided therein and a tubular member with a proximal end and a distal end, wherein the tubular member forms a lumen and is attached to the handle. It further comprises a palpation tool for palpating a pulley of a finger, the palpation tool having a proximal end and an atraumatic distal end, wherein the palpation tool is configured to be slidably disposed in and extracted from the lumen of the tubular member, and an incision tool with a proximal end and a distal end, wherein the incision tool comprises a cutter, wherein the incision tool is configured to be slidably disposed in the lumen of the tubular member, wherein the incision tool is configured to be disposed in the tubular member after extraction of the palpation tool.

The handle and the tubular member may function as a carrier, which can receive the palpation and incision tools. Once the surgical system is inserted under the skin of a patient's hand, the palpation tool may be used for identifying the location of the affected pulley while protecting the surrounding tissue from unnecessary damages. After the position of the pulley has been determined by the palpation tool, e.g. by a noticeable resistance by the tissue, the tubular member may be maintained at the same position, while the palpation tool is extracted from the member. The tubular member may be moved slightly further into the tissue to contact and engage with the pulley. Subsequently, the incision tool is inserted into the tubular member and its cutter is used to sever the pulley by extending the cutter distally out from the distal end of the tubular member. The cut may be created with a sliding motion of the incision tool. Afterwards, the surgical system may be extracted from the patient. In this manner, the operation can be performed more reliably and safely despite being a minimally invasive treatment. The accuracy and success rate of the operation can thus be increased. Compared to an open surgery, the expenses can be reduced because an operation hall is no longer necessary and the device can compensate for the skill of the operator.

Preferably, the tubular member comprises a length graduation, more preferably a millimetre graduation. This may allow for enhanced assessment of the length to which the tubular member has been inserted.

Preferably, the handle comprises at least two openings, wherein a first opening of the handle is positioned at a proximal end of the handle and a second opening of the handle is positioned at a distal end of the handle, wherein the lumen of the handle extends to the openings.

The handle may comprise at least one positioning element configured to limit movement of the palpation tool and/or of the incision tool in the distal direction. Limiting the movement of the tools may prevent damage to the tissue from unintended movements.

Furthermore, the tubular member may have a sharp distal edge at the distal end for inserting the tubular member through the skin. By doing this, no additional tool or scalpel would be necessary to create an adequately sized hole for the insertion of the surgical system into the skin of a patient.

The sharp distal edge may be provided in different shapes known from biopsy needles. Preferably, the tubular member extends along a longitudinal axis, wherein the distal end of the tubular member is C-shaped in a side view perpendicular to the longitudinal axis. Such C-shape is advantageous because it allows to engage the affected pulley to maintain the position of the tubular member when the palpation tool is extracted and/or to lift the pulley when cutting with the incision tool.

More preferably, the distal edge comprises a concave portion. The concave portion may extend from a first end point of the concave portion to a second end point of the concave portion, wherein the first end point is distal from the second end point. An apex of the concave portion may be proximal from the first end point and proximal from the second end point. Preferably, the distal edge extends from a first tip to a second tip, wherein the first tip extends further in the distal direction than the second tip. The first tip may form the first end point and the second tip may form the second end point. Such shape has been found to be particularly advantageous for inserting the tubular member into the skin of the patient and engaging with the affected tissue after the retraction of the palpation tool.

In other words, the distal end of the tubular member may be two-pronged.

According to a preferred embodiment of the present invention, the tubular member does not have slots extending through the sidewall thereof. Thus, the tissue does not get attached to and/or injured by the side walls of the tubular member.

Preferably, the palpation tool is configured to slide between a retracted position and a maximally distally extended position. The sliding motion may be blocked, preferably by the positioning element, at the maximally distally extended position of the palpation tool. This design prevents the palpation tool from extending further than necessary. Furthermore, the palpation tool may comprise a groove along an outer surface thereof, wherein the sliding motion is blocked upon an end of the groove contacting the positioning element at the maximally distally extended position of the palpation tool. Such locking mechanism is easy to manufacture.

Preferably, the distal end of the palpation tool in a maximally distally extended position extends further in the distal direction than the distal end of the tubular member. This may be helpful for protecting the tissue from the distal end of the tubular member when advancing the device through the tissue towards the pulley. The distal end of the palpation tool in a maximally distally extended position may extend at least 0.5 mm further, preferably at least 1 mm further, in the distal direction than the distal end of the tubular member. Preferably, the palpation tool extends less than 10 mm, more preferably less than 5 mm or less than 3 mm, e.g. 1.4 mm, distally past the distal end of the tubular member when in the maximally distally extended position.

Preferably, the distal end of the palpation tool is closed. This may prevent tissue from entering the interior of the palpation tool. More preferably, the distal end of the palpation tool may be rounded and/or dome-shaped. These shapes are beneficial in navigating the palpation tool through the tissue to reach the affected pulley without damaging the tissue.

Similarly, the incision tool may be configured to slide between a retracted position and a maximally distally extended position. The sliding motion may be blocked, preferably by a positioning element, at the maximally distally extended position of the incision tool. This design prevents the incision tool from extending further than necessary. Furthermore, the incision tool may comprise a groove along an outer surface thereof, wherein the sliding motion is blocked upon an end of the groove contacting the positioning element at the maximally distally extended position of the incision tool. Such locking mechanism is easy to manufacture.

Preferably, the distal end of the incision tool in a maximally distally extended position extends further in the distal direction from the distal end of the tubular member than the palpation tool in the maximally distally extended position. This allows the incision to cut through the pulley. The distal end of the incision tool in a maximally distally extended position extends less than 15 mm, preferably less than 10 mm (e.g. 6.8 mm), further in the distal direction than the distal end of the tubular member.

The cutter of the incision tool may be oriented in the distal direction. Preferably, the cutter is a blade. In a preferred embodiment, the cutter is a blade with a semi-circular shape. It may also be a blade comprising a semi-circular cutting-edge portion and/or a longitudinal cutting-edge portion, preferably wherein the radius of the semi-circular cutting-edge portion is less than 5 mm or less than 2 mm, preferably less than 1.5 mm (e.g. 1.2 mm), and/or the length of the longitudinal cutting-edge portion is less than 15 mm, preferably less than 10 mm, such as equal to or less than 5 mm. Furthermore, the cutter may be a blade with a V-shape or a blade with an elliptical shape. These shape features are considered to be appropriate and effective in cutting through the affected pulley.

The proximal end of the palpation tool may be coupled to a palpation tool actuator. This allows the operator a better grip and improves the usability of the device. The proximal end of the palpation tool may be coupled to the palpation tool actuator by a threaded connection or form fit and/or friction fit or adhesion. Moreover, the proximal end of the palpation tool may be an integral part of the palpation tool actuator. The palpation tool actuator may be configured to be grasped by a user and manually pushed in the distal direction. A push motion can be controlled easily and accurately.

The surgical system may further comprise an elastic member that biases the palpation tool in the proximal direction when the palpation tool is moved in the distal direction. This additional feature is beneficial because the tool is only in motion if the operator presses the palpation tool on purpose. Preferably, the elastic member is a coil spring disposed about a proximal end portion of the palpation tool. It is an inexpensive but stable mechanism, which improves the safety of the operation.

Preferably, the palpation tool is rigid and/or the palpation tool is substantially solid. This may avoid errors when palpating.

According to a second configuration of the present invention, a surgical system for minimally invasive treatment of a trigger finger comprises a handle with a lumen provided therein. It further comprises a tubular member for palpating a pulley of a finger, the tubular member having a proximal end and an atraumatic distal end, wherein the tubular member forms a lumen and is attached to the handle; and an incision tool with a proximal end and a distal end, wherein the incision tool comprises a cutter, wherein the incision tool is configured to be slidably disposed in the lumen of the tubular member.

The system may further comprise a pre-incision tool for cutting an insertion hole in the skin through which the tubular member may be inserted.

The handle and the tubular member may function as a carrier, which can receive the incision tool.

Once the surgical system is under the skin of a patient's hand or foot, the tubular member can serve as a palpation tool for identifying the location of the affected tissue. The surrounding tissue is protected by the atraumatic distal end from unnecessary damages. When the position of the pulley has been determined by contacting it with the tubular member, which can be a noticeable resistance by the tissue, the tubular member can be moved over or under the pulley to engage the pulley therewith. In this manner, the affected pulley can be safely located and held. Subsequently, the incision tool may be inserted into the tubular member and its cutter is used to sever the pulley in a controlled and safe manner. Afterwards, the surgical system is extracted from the hand or the foot of the patient.

Preferably, the handle comprises at least one opening, wherein a first opening of the handle is positioned on a distal end of the handle.

The handle may comprise at least one positioning element configured to limit movement of the incision tool in the distal direction. The incision tool can be controlled in its movement to prevent a possible damage to the tissues from unintended movements.

In a preferred embodiment, the distal end of the tubular member is closed. This may prevent tissue from entering the interior of the tubular member. More preferably, the distal end of the tubular member is rounded and/or dome-shaped. These shapes are beneficial in navigating the tubular member through the tissue to reach the affected pulley without damaging the tissue.

Preferably, the tubular member may have a side opening to its lumen along a distal portion of the tubular member. Such opening is helpful for engaging the tubular member with the pulley. Furthermore, a distal end of the side opening may be hook-shaped. More preferably, the hook shape forms a recess in the tubular member that is open in a proximal direction to engage the pulley. The tubular member may be moved in the distal direction to pass it over or under the pulley. The tubular member may then be withdrawn proximally, thereby engaging said pulley with the hook.

The distal end of the side opening may be positioned at a length at least 10 mm away from the distal end of the tubular member. The side opening may have a length of at least 5 mm, preferably at least 6 mm, and/or the side opening may have a length of less than 20 mm or less than 15 mm, preferably less than 10 mm.

Preferably, the incision tool is configured to slide between a retracted position and a maximally distally extended position, wherein the sliding motion is blocked, preferably by the positioning element, at the maximally distally extended position of the incision tool. This design prevents the incision tool from extending further than necessary. Furthermore, the incision tool may comprise a groove along an outer surface thereof, wherein the sliding motion is blocked upon an end of the groove contacting the positioning element at the maximally distally extended position of the incision tool.

At least a portion of a cutting edge of the cutter may face outwardly in a direction oblique or perpendicular to a longitudinal axis of the tubular member, wherein the system may be configured such that said portion of the cutter is exposed along the side opening. Another portion of the cutter may face in the direction of the longitudinal axis. Preferably, the cutter of the incision tool is a blade. In a preferred embodiment, the cutter is a blade with a semi-circular shape. It may also be a blade comprising a semi-circular cutting-edge portion and/or a longitudinal cutting-edge portion, preferably wherein the radius of the semi-circular cutting-edge portion is less than 5 mm or less than 2 mm, preferably less than 1.5 mm (such as equal or less than 1.2 mm), and/or the length of the longitudinal cutting-edge portion is less than 15 mm or less than 10 mm, preferably less than 8 mm (such as equal to or less than 5 mm). Furthermore, the cutter may be a blade with a V-shape or a blade with an elliptical shape. Such blades are considered to be appropriate and effective in cutting through the affected pulley.

Referring now to both the first and the second configurations indicated above, a distal end of the handle may comprise a supporting body. Furthermore, the supporting body may taper in the distal direction. The supporting body can improve the flow of forces from the tubular member into the handle and vice versa.

At least one positioning element may project into the lumen of the handle and/or the lumen of the tubular member. Preferably, the positioning element is movable in a direction perpendicular to a longitudinal axis of the lumen of the tubular member. More preferably, the positioning element comprises a screw or an integral part of the handle. This provides a cost-effective solution to attach a positioning element, which limits the movement of the tools within the surgical system. The positioning element may be positioned at a length less than 50 mm, preferably less than 40 mm, away from the opening on the distal end of the handle. The length may determine, together with the groove, how far the tools can extend from the tubular member.

In preferred embodiments, the tubular member is fixed to the handle by a fixation member extending through the handle. The fixation member may project into the lumen of the handle, preferably wherein the fixation member is a screw. Alternatively, the tubular member may be fixed to the handle by adhesion. The fixation of the tubular member improves the overall stability of the surgical system during the operation. Generally, it is easier to manufacture the tubular member and handle separately and assemble them together afterwards. A portion of the tubular member may extend through the handle and the tubular member may be fixed to the handle along said portion. Alternatively, the tubular member may be an integral part of the handle.

The distal end of the tubular member may be spaced from the opening on the distal end of the handle at least 10 mm, preferably at least 15 mm or at least 20 mm, and/or less than 100 mm or less than 50 mm, preferably less than 40 mm or less than 30 mm. Such lengths have been found to be advantageous for reaching the pulley.

Preferably, the outer diameter of the tubular member is less than 3 mm and/or more than 2 mm. Preferably, the tubular member is rigid and/or the tubular member is substantially solid. This may avoid positioning errors.

The cutter of the incision tool may be positioned about a distal end portion of the incision tool. Preferably, the cutter is attached to the incision tool by adhesion. Alternatively, it may be an integral part of the incision tool. Furthermore, the cutter may be an ultrasonic blade or a hot filament or an electrosurgical device.

The proximal end of the incision tool may be coupled to an incision tool actuator. This allows the operator a better grip and improves the usability of the device. Furthermore, the steadiness from using an actuator makes the operation safer and more reliable. The proximal end of the incision tool may be coupled to the incision tool actuator by a threaded connection or form fit and/or friction fit or adhesion. Moreover, the proximal end of the incision tool may be integral with the incision tool actuator. The incision tool actuator may be configured to be grasped by a user and manually pushed in the distal direction. A push motion can be controlled more easily and accurately than e.g. a pulling motion.

The surgical system may further comprise an elastic member that biases the incision tool in the proximal direction when the incision tool is moved in the distal direction. This additional feature is beneficial because the tool is only in motion if the operator presses the incision tool on purpose. Preferably, the elastic member is a coil spring disposed about a proximal end portion of the incision tool. It is an inexpensive but stable mechanism, which improves the safety of the operation.

Preferably, the incision tool is rigid and/or the incision tool is substantially solid. The stability of the tool can make a positive contribution to overall reliability, safety and usability of the surgical system.

Another aspect of the present application relates to a method for minimally invasive treatment of a trigger finger, the method comprising: inserting a tubular member through the skin, inserting a palpation tool with an atraumatic distal tip through the tubular member and protruding the atraumatic tip out of the tubular member by a first length, palpating a pulley by advancing the tubular member and/or the palpation tool tip through the skin, extracting the palpation tool from the tubular member, advancing an incision tool through the tubular member and extending the incision tool out of the tubular member by a second length that is larger than the first length, thereby cutting into the pulley, extracting the incision tool, and extracting the tubular member. Preferably, the tubular member engages the pulley with its distal end as discussed for the first configuration of the surgical system.

In one embodiment, at least one positioning element of the handle prevents the distal end of the palpation tool, when fully inserted, from extending more than 10 mm, more than 5 mm, or more than 2 mm further in the distal direction than the distal end of the tubular member. Furthermore, at least one positioning element of the handle (which may be the same or a different positioning element than the one for the palpation tool) may prevent the distal end of the incision tool, when fully inserted, from extending more than 10 mm, e.g. equal to or less than 6.8 mm, further in the distal direction than the distal end of the tubular member.

According to another surgical method for minimally invasive treatment of a trigger finger, which is particularly beneficial in the context of the second configuration of the surgical system described above, the method comprises: inserting a tubular member with an atraumatic distal tip through an insertion hole of the skin, palpating a pulley by advancing the tubular member through the skin, cutting into the pulley by advancing an incision tool through the tubular member, extracting the incision tool, and extracting the tubular member. Preferably, the tubular member has a side opening that is used to engage the pulley, as discussed for the second configuration of the surgical system above.

### BRIEF DESCRIPTION OF THE DRAWINGS

The invention will be described in more detail with reference to the figures below. These figures disclose embodiments of the invention for illustrational purposes only. In particular, the disclosure provided by the figures is not meant to limit the scope of protection conferred by the invention.

FIG. 1 is a perspective view of a surgical system for minimally invasive treatment of a trigger finger, in accordance with an embodiment of the present invention.

FIG. 2 is a cross section side view of the surgical system of FIG. 1 with a palpation tool in a maximally distally extended position.

FIG. 3a is a cross section side view of the surgical system of FIG. 1 with an incision tool in a proximally retracted position.

FIG. 3b is a cross section side view of the surgical system of FIG. 1 with the incision tool in a maximally distally extended position.

FIG. 3c is an enlarged side view of a distal end of the surgical system of FIG. 1 with the incision tool in a maximally distally extended position.

FIG. 4 is a perspective view of a surgical system for minimally invasive treatment of a trigger finger, in accordance with another embodiment of the present invention.

FIG. 5a is a cross section side view of the surgical system of FIG. 4.

FIG. 5b is an enlarged cross section side view of the surgical system of FIG. 4.

FIG. 6a is an enlarged cross section side view of the distal end of the surgical system of FIG. 4 with an incision tool in a proximally retracted position.

FIG. 6b is an enlarged cross section side view of a distal end of the surgical system of FIG. 4 with an incision tool in a maximally distally extended position.

### DETAILED DESCRIPTION

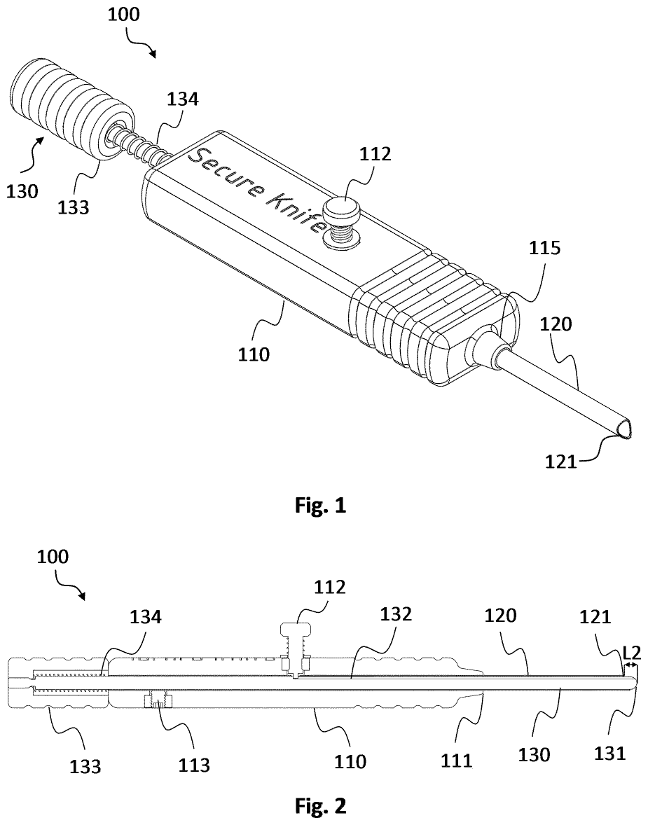

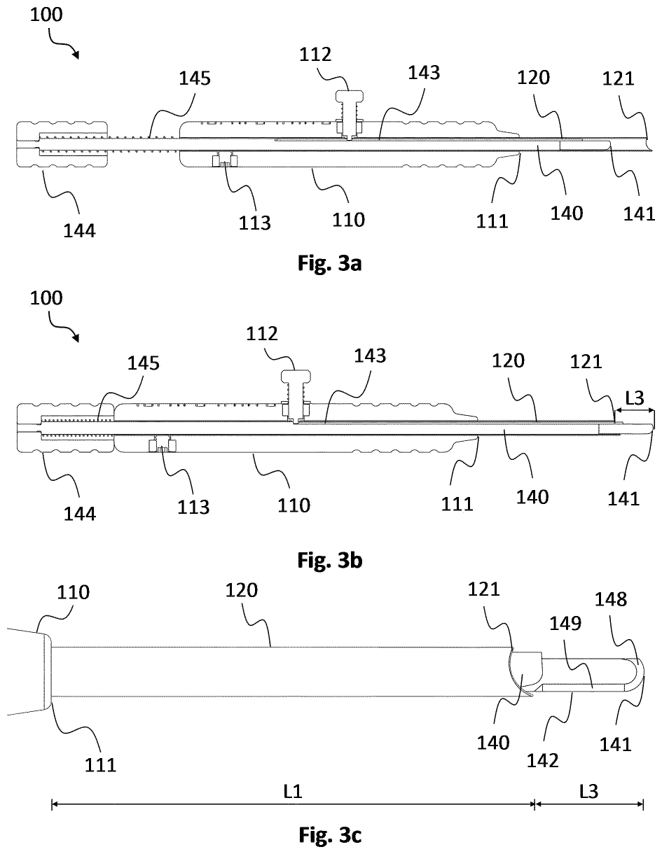

FIG. 1 schematically illustrates a first configuration of the present invention, which shows a surgical system 100 comprising a handle 110, wherein a tubular member 120 is attached to the handle 110. A lumen of the tubular member 120 is coaxial with a lumen of the handle 110. Furthermore, the tubular member 120 has a distal end 121, which has a sharp distal edge. The handle 110 further comprises a positioning element 112. A palpation tool 130 is inserted into the lumen of the tubular member 120. A palpation tool actuator 133 is located at the proximal end of the palpation tool 130, while an elastic member 134 is positioned adjacent a proximal end portion of the palpation tool 130. The elastic member 134 biases the palpation tool 130 in the proximal direction when the palpation tool 130 is moved in the distal direction. The palpation tool 130 disposed in the tubular member 120 is shown in a retracted position in FIG. 1. In this embodiment, the distal end 121 with the sharp distal edge preferably has a concave portion, which can be used to insert the surgical system 100 into the skin of a patient and later engage with the affected pulley of the patient. The concave portion may have a C-shape (see FIG. 3c).

FIG. 2 shows a cross section side view of FIG. 1 in detail. Unlike FIG. 1, the palpation tool 130 within the tubular member 120 is in a maximally distally extended position. In this position, an atraumatic distal end 131 of the palpation tool 130 is further extended distally than the distal end 121 of the tubular member 120 by a length L2, thereby protecting the tissues from the distal end 121 of the tubular member 120 after insertion through the skin and while advancing the tubular member 120 distally towards the pulley. The length L2 may be, for example, more than 1 mm, preferably more than 1.4 mm, and/or less than 10 mm, more preferably less than 5 mm.

Preferably, the maximally distally extended position is reached when the sliding motion is blocked upon an end of a groove 132 of the palpation tool 130 contacting the positioning element 112.

The tubular member 120 may be fixed to the handle 110 by a fixation member 113 projecting into the lumen of the handle 110.

FIGS. 3a and 3b show cross section side views of the system 100 after extracting the palpation tool 130 and with an incision tool 140 slidably disposed in the tubular member 120.

In FIG. 3a, the incision tool 140 is positioned in a retracted position. Similar to the palpation tool 130, an incision tool actuator 144 is located at the proximal end of the incision tool 140, while an elastic member 145 may be positioned adjacent a proximal end portion of the incision tool 140. The elastic member 145 biases the incision tool 140 in the proximal direction when the incision tool 140 is moved in the distal direction.

In FIG. 3b, the incision tool 140 within the tubular member 120 is in a maximally distally extended position. The maximally distally extended position may be reached when the sliding motion is blocked upon an end of a groove 143 of the incision tool 140 contacting the positioning element 112. In this position, a distal end 141 of the incision tool 140 extends distally from the distal end 121 of the tubular member 120. In particular, the incision tool 140 may extend distally past the tubular member 120 by a length L3. The length L3 may be less than 10 mm, e.g. equal to or less than 6.8 mm.

FIG. 3c shows an enlarged side view of the distal end of the surgical system 100, especially the distal end 141 of the incision tool 140. The cutter 142 is attached to the distal portion 141 of the incision tool 140. In this embodiment, the cutter 142 comprises a semi-circular cutting-edge portion 148 and a longitudinal cutting-edge portion 149.

The distal end 111 of the handle 110 may comprise a supporting body 115 tapering in the distal direction (see FIG. 1).

As shown in FIG. 3c, the tubular member may extend distally from the handle 110 by a length L1. The length L1 may be less than 70 mm, preferably less than 40 mm.

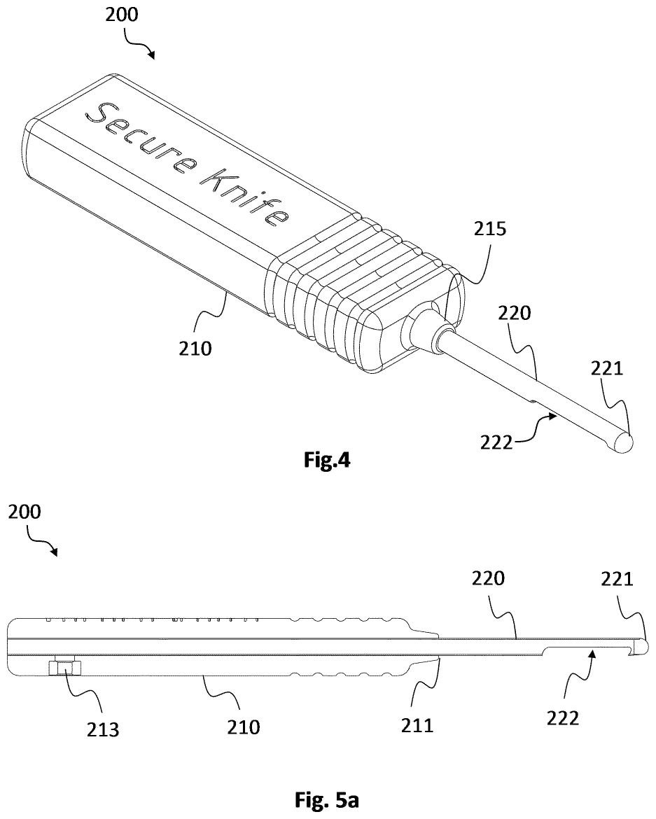

FIG. 4 schematically illustrates a second configuration of the present invention, which shows a surgical system 200 comprising a handle 210, wherein a tubular member 220 is attached to the handle 210. A lumen of the tubular member 220 is coaxial with a lumen of the handle 210. The tubular member 220 has an atraumatic distal end 221. The handle 210 may further comprise a fixation member 213, as previously described. In this embodiment, the tubular member 120 has a side opening 222, which can be used to engage with the affected pulley of the patient. The surgical system may further comprise a pre-incision tool, which may be used to create an insertion hole through the skin, which is not illustrated in the figures.

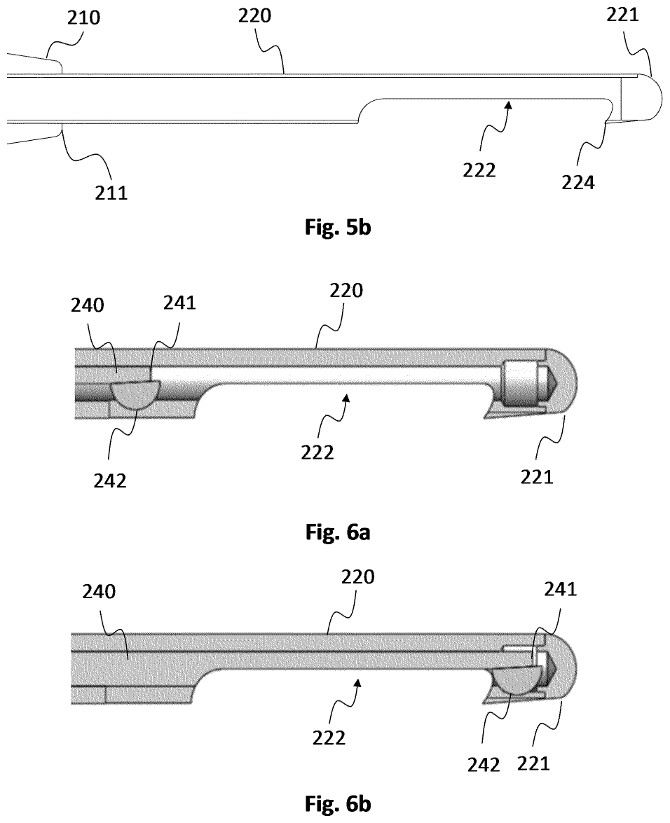

FIG. 5a shows a cross section side view of FIG. 4. The incision tool 240 (see FIGS. 6a and 6b) can be positioned in its retracted position and be pushed into a maximally distally extended position. An incision tool actuator (not shown) may be located at the proximal end of the incision tool 240, while an elastic member (not shown) may be positioned adjacent a proximal end portion of the incision tool 240 (as described for the first configuration). The elastic member may bias the incision tool 240 in the proximal direction when the incision tool 240 is moved in the distal direction. The distal end 241 of the incision tool 240 extends close to the distal end 221 of the tubular member 220.

FIG. 5b shows an enlarged cross section side view of the distal end of the surgical system 200, especially the distal end 221 of the tubular member 220. As shown in FIG. 5b, a hook 224 for engaging the pulley is formed along the side opening 222, preferably at a distal end of the side opening 222. The hook 224 is open towards the proximal direction. This allows to engage the pulley by a proximal movement of the tubular member 220.

FIGS. 6a and 6b show enlarged cross section side views of the distal end of the surgical system 200, especially the distal end 241 of the incision tool 240. The cutter 242 is attached to the distal portion 241 of the incision tool 240. In this embodiment, the cutter 242 may comprise a semi-circular cutting-edge portion and/or a longitudinal cutting-edge portion. Furthermore, the distal end of the handle 211 may comprise a supporting body 215 tapering in the distal direction.

The invention may be defined, for example, by the following aspects:

1. A surgical system for minimally invasive treatment of a trigger finger comprising:
a handle with a lumen provided therein;
a tubular member with a proximal end and a distal end, wherein the tubular member forms a lumen and is attached to the handle;
a palpation tool for palpating a pulley of a finger, the palpation tool having a proximal end and an atraumatic distal end, wherein the palpation tool is configured to be slidably disposed in and extracted from the lumen of the tubular member; and
an incision tool with a proximal end and a distal end, wherein the incision tool comprises a cutter, wherein the incision tool is configured to be slidably disposed in the lumen of the tubular member, wherein the incision tool is configured to be disposed in the tubular member after extraction of the palpation tool.
   2. A surgical system according to aspect 1,
wherein the handle comprises at least two openings, wherein a first opening of the handle is positioned on a proximal end of the handle and a second opening of the handle is positioned on a distal end of the handle, wherein the lumen of the handle extends to the openings.
   3. A surgical system according to aspect 1 or 2,
wherein the handle comprises at least one positioning element configured to limit movement of the palpation tool and/or of the incision tool in the distal direction.
   4. A surgical system according to any one of aspects 1-3,
wherein the tubular member has a sharp distal edge at the distal end for inserting the tubular member through the skin.
   5. A surgical system according to any one of aspects 1-4,
wherein the tubular member extends along a longitudinal axis, wherein the distal end of the tubular member is C-shaped in a side view perpendicular to the longitudinal axis.
   6. A surgical system according to any one of aspects 4-5,
wherein the distal edge comprises a concave portion.
   7. A surgical system according to aspect 6,
wherein the concave portion extends from a first end point of the concave portion to a second end point of the concave portion, wherein the first end point is distal from the second end point.
   8. A surgical system according to aspect 7,
wherein an apex of the concave portion is proximal from the first end point and proximal from the second end point.
   9. A surgical system according to any one of aspects 6-8,
wherein the distal edge extends from a first tip to a second tip, wherein the first tip extends further in the distal direction than the second tip.
   10. A surgical system according to aspect 9,
wherein the first tip forms the first end point and the second tip forms the second end point.
   11. A surgical system according to any one of aspects 1-10,
wherein the distal end of the tubular member is two-pronged.
   12. A surgical system according to any one of aspects 1-10,
wherein the tubular member does not have slots extending through the sidewall thereof.
   13. A surgical system according to any one of aspects 1-12,
wherein the palpation tool is configured to slide between a retracted position and a maximally distally extended position, wherein the sliding motion is blocked, preferably by the positioning element, at the maximally distally extended position of the palpation tool.
   14. A surgical system according to aspect 13,
wherein the palpation tool comprises a groove along an outer surface thereof, wherein the sliding motion is blocked upon an end of the groove contacting the positioning element at the maximally distally extended position of the palpation tool.
   15. A surgical system according to any one of aspects 1-14,
wherein the distal end of the palpation tool in a maximally distally extended position extends further in the distal direction than the distal end of the tubular member.
   16. A surgical system according to aspect 15,
wherein the distal end of the palpation tool in a maximally distally extended position extends at least 1 mm further, preferably at least 1.4 mm further, in the distal direction than the distal end of the tubular member.
   17. A surgical system according to any one of aspects 1-16,
wherein the distal end of the palpation tool is closed.
   18. A surgical system according to any one of aspects 1-17,
wherein the distal end of the palpation tool is rounded.
   19. A surgical system according to any one of aspects 1-18,
wherein the distal end of the palpation tool is dome-shaped.
   20. A surgical system according to any one of aspects 1-19,
wherein the incision tool is configured to slide between a retracted position and maximally distally extended position, wherein the sliding motion is blocked, preferably by the positioning element, at the maximally distally extended position of the incision tool.
   21. A surgical system according to aspect 20,
wherein the incision tool comprises a groove along an outer surface thereof, wherein the sliding motion is blocked upon an end of the groove contacting the positioning element at the maximally distally extended position of the incision tool.
   22. A surgical system according to aspect 20 or 21,
wherein the distal end of the incision tool in a maximally distally extended position extends further in the distal direction from the distal end of the tubular member than the palpation tool in the maximally distally extended position.
   23. A surgical system according to any one of aspects 20-22,
wherein the distal end of the incision tool in a maximally distally extended position extends less than 15 mm or less than 10 mm, preferably equal or less than 6.8 mm, further in the distal direction than the distal end of the tubular member.
   24. A surgical system according to any one of aspects 1-23,
wherein the cutter of the incision tool is oriented in the distal direction.
   25. A surgical system according to any one of aspects 1-24,
wherein the cutter is a blade.
   26. A surgical system according to aspect 25,
wherein the cutter is a blade with a semi-circular shape.
   27. A surgical system according to aspect 25,
wherein the cutter is a blade comprising a semi-circular cutting-edge portion and/or a longitudinal cutting-edge portion, preferably wherein the radius of the semi-circular cutting-edge portion is less than 5 mm, less than 3 mm or less than 1.5 mm, preferably equal or less than 1.2 mm, and/or the length of the longitudinal cutting-edge portion is less than 15 mm, less than 10 mm or less than 8 mm.
   28. A surgical system according to aspect 25,
wherein the cutter is a blade with a V-shape.
   29. A surgical system according to aspect 25,
wherein the cutter is a blade with an elliptical shape.
   30. A surgical system according to any one of aspects 1-29,
wherein the proximal end of the palpation tool is coupled to a palpation tool actuator.
   31. A surgical system according to aspect 30,
wherein the proximal end of the palpation tool is coupled to the palpation tool actuator by a threaded connection.
   32. A surgical system according to aspect 30,
wherein the proximal end of the palpation tool is coupled to the palpation tool actuator by form fit and/or friction fit.
   33. A surgical system according to aspect 30,
wherein the proximal end of the palpation tool is coupled to the palpation tool actuator by adhesion.
   34. A surgical system according to aspect 30,
wherein the proximal end of the palpation tool is an integral part of the palpation tool actuator.
   35. A surgical system according to any one of aspects 1-34,
wherein the palpation tool actuator is configured to be grasped by a user and manually pushed in the distal direction.
   36. A surgical system according to any one of aspects 1-35,
further comprising an elastic member that biases the palpation tool in the proximal direction when the palpation tool is moved in the distal direction.
   37. A surgical system according to aspect 36,
wherein the elastic member is a coil spring disposed about a proximal end portion of the palpation tool.
   38. A surgical system according to any one of aspects 1-37,
wherein the palpation tool is rigid.
   39. A surgical system according to any one of aspects 1-38,
wherein the palpation tool is substantially solid.
   40. A surgical system for minimally invasive treatment of a trigger finger comprising:
a handle with a lumen provided therein;
a tubular member for palpating a pulley of a finger, the tubular member having a proximal end and an atraumatic distal end, wherein the tubular member forms a lumen and is attached to the handle; and
an incision tool with a proximal end and a distal end, wherein the incision tool comprises a cutter, wherein the incision tool is configured to be slidably disposed in the lumen of the tubular member.
   41. A surgical system according to aspect 40,
wherein the handle comprises at least one opening, wherein a first opening of the handle is positioned on a distal end of the handle.
   42. A surgical system according to aspect 40 or 41,
wherein the handle comprises at least one positioning element configured to limit movement of the incision tool in the distal direction.
   43. A surgical system according to any one of aspects 40-42,
wherein the distal end of the tubular member is closed.
   44. A surgical system according to any one of aspects 40-43,
wherein the distal end of the tubular member is rounded.
   45. A surgical system according to any one of aspects 40-44,
wherein the distal end of the tubular member is dome-shaped.
   46. A surgical system according to any one of aspects 40-45,
wherein the tubular member has a side opening to its lumen along a distal portion of the tubular member.
   47. A surgical system according to aspect 46,
wherein a distal end of the side opening is hook-shaped.
   48. A surgical system according to aspect 47,
wherein the hook shape forms a recess in the tubular member that is open in a proximal direction to engage the pulley.
   49. A surgical system according to any one of aspects 46-48,
wherein the distal end of side opening is positioned at a length at least 10 mm away from the distal end of the tubular member.
   50. A surgical system according to any one of aspects 46-49,
wherein the side opening has a length of at least 5 mm, preferably at least 6 mm, and/or wherein the side opening has a length of less than 15 mm, preferably less than 10 mm.
   51. A surgical system according to any one of aspects 40-50,
wherein the incision tool can slide between a retracted position and maximally distally extended position, wherein the sliding motion is blocked, preferably by a positioning element, at the maximally distally extended position of the incision tool.
   52. A surgical system according to aspect 51,
wherein the incision tool comprises a groove along an outer surface thereof, wherein the sliding motion is blocked upon an end of the groove contacting the positioning element at the maximally distally extended position of the incision tool.
   53. A surgical system according to any one of aspects 46-52,
wherein at least a portion of the cutter faces in a direction perpendicular to a longitudinal axis of the tubular member, wherein the system is configured such that said portion of the cutter may be exposed along the side opening, and wherein another portion of the cutter faces in the direction of the longitudinal axis.
   54. A surgical system according to any one of aspects 40-53,
wherein the cutter of the incision tool is a blade.
   55. A surgical system according to aspect 54,
wherein the cutter is a blade with a semi-circular shape.
   56. A surgical system according to aspect 54,
wherein the cutter is a blade comprising a semi-circular cutting-edge portion and/or a longitudinal cutting-edge portion, preferably wherein the radius of the semi-circular cutting-edge portion is less than 5 mm, less than 3 mm or less than 1.5 mm, and/or the length of the longitudinal cutting-edge portion is less than 15 mm, less than 10 mm, or less than 8 mm.
   57. A surgical system according to aspect 54,
wherein the cutter is a blade with a V-shape.
   58. A surgical system according to aspect 54,
wherein the cutter is a blade with an elliptical shape.
   59. A surgical system according to any one of aspects 1-58,
wherein a distal end of the handle comprises a supporting body.
   60. A surgical system according to aspect 59,
wherein the supporting body tapers in the distal direction.
   61. A surgical system according to any one of aspects 3-39 or 42-58,
wherein the at least one positioning element projects into the lumen of the handle and/or the lumen of the tubular member.
   62. A surgical system according to aspect 61,
wherein the positioning element is movable in a direction perpendicular to a longitudinal axis of the lumen of the tubular member.
   63. A surgical system according to aspect 61 or 62,
wherein the positioning element comprises a screw.
   64. A surgical system according to aspect 61 or 62,
wherein the positioning element is an integral part of the handle.
   65. A surgical system according to any one of aspects 61-64,
wherein the positioning element is positioned at a length less than 50 mm, preferably less than 40 mm away, from the opening on the distal end of the handle.
   66. A surgical system according to any one of aspects 1-65,
wherein the tubular member is fixed to the handle by a fixation member extending through the handle.
   67. A surgical system according to aspect 66,
wherein the fixation member projects into the lumen of the handle, preferably wherein the fixation member is a screw.
   68. A surgical system according to any one of aspects 1-67,
wherein the tubular member is fixed to the handle by adhesion.
   69. A surgical system according to any one of aspects 1-68,
wherein the tubular member is fixed to the handle, preferably wherein a portion of the tubular member extends through the handle and the tubular member is fixed to the handle along said portion; or
wherein the tubular member is an integral part of the handle.
   70. A surgical system according to any one of aspects 1-69,
wherein the distal end of the tubular member is spaced from the opening on the distal end of the handle at least 10 mm.
   71. A surgical system according to aspect 70,
wherein the distal end of the tubular member is spaced from the opening on the distal end of the handle at least 15 mm.
   72. A surgical system according to any one of aspects 1-71,
wherein the distal end of the tubular member is spaced from the opening on the distal end of the handle less than 100 mm or less than 50 mm.
   73. A surgical system according to aspect 72,
wherein the distal end of the tubular member is spaced from the opening on the distal end of the handle less than 30 mm.
   74. A surgical system according to any one of aspects 1-73,
wherein the outer diameter of the tubular member is less than 3 mm.
   75. A surgical system according to aspect 74,
wherein the outer diameter of the tubular member is more than 2 mm.
   76. A surgical system according to any one of aspects 1-75,
wherein the tubular member is rigid.
   77. A surgical system according to any one of aspects 1-76,
wherein the tubular member is substantially solid.
   78. A surgical system according to any one of aspects 1-77,
wherein the cutter of the incision tool is positioned about a distal end portion of the incision tool.
   79. A surgical system according to aspect 78,
wherein the cutter is attached to the incision tool by adhesion.
   80. A surgical system according to aspect 78,
wherein the cutter is an integral part of the incision tool.
   81. A surgical system according to aspect 78,
wherein the cutter is an ultrasonic blade.
   82. A surgical system according to aspect 78,
wherein the cutter is a hot filament.
   83. A surgical system according to aspect 78,
wherein the cutter is an electrosurgical device.
   84. A surgical system according to any one of aspects 1-83,
wherein the proximal end of the incision tool is coupled to an incision tool actuator.
   85. A surgical system according to aspect 84,
wherein the proximal end of the incision tool is coupled to incision tool actuator by a threaded connection.
   86. A surgical system according to aspect 84,
wherein the proximal end of the incision tool is coupled to the incision tool actuator by form fit and/or friction fit.
   87. A surgical system according to aspect 84,
wherein the proximal end of the incision tool is coupled to the incision tool actuator by adhesion.
   88. A surgical system according to aspect 84,
wherein the proximal end of the incision tool is an integral part of the incision tool actuator.
   89. A surgical system according to any one of aspects 1-88,
wherein the incision tool actuator is configured to be grasped by a user and manually pushed in the distal direction.
   90. A surgical system according to aspect 89,
further comprising an elastic member that biases the incision tool in the proximal direction when the incision tool is moved in the distal direction.
   91. A surgical system according to aspect 90,
wherein the elastic member is a coil spring disposed about a proximal end portion of the incision tool.
   92. A surgical system according to any one of aspects 1-91,
further comprising a pre-incision tool for cutting an insertion hole.
   93. A surgical system according to any one of aspects 1-92,
wherein the incision tool is rigid.
   94. A surgical system according to any one of aspects 1-93,
wherein the incision tool is substantially solid.
   95. A method for minimally invasive treatment of a trigger finger comprising:
inserting a tubular member through the skin,
inserting a palpation tool with an atraumatic distal tip through the tubular member and protruding the atraumatic tip out of the tubular member by a first length,
palpating a pulley by advancing the tubular member with the protruded atraumatic tip through the skin,
extracting the palpation tool from the tubular member,
advancing an incision tool through the tubular member and extending the incision tool out of the tubular member by a second length that is larger than the first length, thereby cutting into the pulley,
extracting the incision tool, and
extracting the tubular member.
   96. The method of aspect 95,
wherein at least one positioning element of the handle prevents the distal end of the palpation tool from extending more than 10 mm, more than 5 mm, more than 3 mm or more than 2 mm further in the distal direction than the distal end of the tubular member.
   97. The method of aspect 95 or 96,
wherein at least one positioning element of the handle prevents the distal end of the incision tool from extending more than 15 mm or more than 10 mm further in the distal direction than the distal end of the tubular member.
   98. A method for minimally invasive treatment of a trigger finger comprising:
inserting a tubular member with an atraumatic distal tip through an insertion hole of the skin,
palpating a pulley by advancing the tubular member through the skin,
cutting into the pulley by advancing an incision tool through the tubular member,
extracting the incision tool, and
extracting the tubular member.

1. A surgical system for minimally invasive treatment of a trigger finger, the system comprising:
a handle with a lumen provided therein;
a tubular member with a proximal end and a distal end, wherein the tubular member forms a lumen and is attached to the handle;
a palpitation tool for palpating a pulley of a finger, that palpitation tool having a proximal end and an atraumatic distal end, wherein the palpitation tool is configured to be slidably disposed in and extracted from the lumen of the tubular member; and
an incision tool with a proximal end and a distal end, wherein the incision tool comprises a cutter, wherein the incision tool is configured to be slidably disposed in the lumen of the tubular member, wherein the incision tool is configured to be disposed in the tubular member after the extraction of the palpitation tool.

2. The surgical system according to claim 1, wherein the tubular member has a sharp distal edge at the distal end for inserting the tubular member through the skin.

3. The surgical system according to claim 1, wherein the distal end of the palpitation tool in a maximally distally extended position extends further in the distal direction that the distal end of the tubular member.

4. The surgical system according to claim 3, wherein the distal end of the incision tool in a maximally distally extended position extends further in the distal direction from the distal end of the tubular member than the palpation tool in the maximally distally extended position, and wherein the cutter of the incision tool is oriented in the distal direction.

5. (canceled)

6. The surgical system according to claim 3, the system further comprising an elastic member that biases the palpation tool in the proximal direction when the palpation tool is moved in the distal direction.

7. The surgical system according to claim 1, wherein the palpitation tool is rigid.

8-12. (canceled)

13. The surgical system according to claim 1, wherein the tubular member is fixed to the handle by a fixation member extending through the handle.

14. The surgical system according to claim 1, the system further comprising an elastic member that biases the incision tool in the proximal direction when the incision tool is moved in the distal direction.

15. The surgical system according claim 1, the system further comprising a pre-incision tool for cutting an insertion hole.

16. A method for minimally invasive treatment of a trigger finger comprising:
inserting a tubular member through the skin,
inserting a palpation tool with an atraumatic distal tip through the tubular member and protruding the atraumatic tip out of the tubular member by a first length,
palpating a pulley by advancing the tubular member with the protruded atraumatic tip through the skin,
extracting the palpation tool from the tubular member,
advancing an incision tool through the tubular member and extending the incision tool out of the tubular member by a second length that is larger than the first length, thereby cutting into the pulley,
extracting the incision tool, and
extracting the tubular member.

17. A surgical system for minimally invasive treatment of a trigger finger, the system comprising:
a handle with a lumen provided therein;
a tubular member for palpating a pulley of a finger, the tubular member having a proximal end and an atraumatic distal end, wherein the tubular member forms a lumen and is attached to the handle; and
an incision tool with a proximal end and a distal end, wherein the incision tool comprises a cutter, wherein the incision tool is configured to be slidably disposed in the lumen of the tubular member.

18. The surgical system according to claim 17, wherein the tubular member has a side opening to its lumen along a distal portion of the tubular member.

19. The surgical system according to claim 18, wherein a distal end of the side opening is hook-shaped, wherein the hook shape forms a recess in the tubular member that is open in a proximal direction to engage the pulley.

20. The surgical system according to claim 18, wherein the incision tool is configured to slide between a retracted position and a maximally distally extended position, wherein the sliding motion is blocked.

21. The surgical system according to claim 20, wherein the sliding motion is blocked by a positioning element, at the maximally distally extended position of the incision tool.

22. The surgical system according to claim 18, wherein at least a portion of the cutter faces in a direction perpendicular to a longitudinal axis of the tubular member, wherein the system is configured such that said portion of the cutter may be exposed along the side opening, and wherein another portion of the cutter faces in the direction of the longitudinal axis.

23. The surgical system according to claim 17, wherein the tubular member is fixed to the handle by a fixation member extending through the handle.

24. The surgical system according to claim 17, the system further comprising an elastic member that biases the incision tool in the proximal direction when the incision tool is moved in the distal direction.

25. The surgical system according to claim 17, the system further comprising a pre-incision tool for cutting an insertion hole.

26. A method for minimally invasive treatment of a trigger finger comprising:
inserting a tubular member with an atraumatic distal tip through an insertion hole of the skin,
palpating a pulley by advancing the tubular member through the skin,
cutting into the pulley by advancing an incision tool through the tubular member,
extracting the incision tool, and
extracting the tubular member.
